(19) United States
(12) Patent Application Publication
(10) Pub. No.: US 2026/0158098 A1
(43) Pub. Date: Jun. 11, 2026

(54) ETIOLATED GREEN TEA POLYSACCHARIDE AND ITS EXTRACTION METHOD AND APPLICATION

(71) Applicants: Zhejiang University of Technology, Hangzhou (CN); Shaoxing Research Institute of Zhejiang University of Technology, Shaoxing (CN)

(72) Inventors: Baoming Tian, Hangzhou (CN); Xue Zhou, Hangzhou (CN)

(21) Appl. No.: 19/407,127

(22) Filed: Dec. 3, 2025

(30) Foreign Application Priority Data
Dec. 10, 2024 (CN) 202411806105.6

(51) Int. Cl. A61K 36/82; A61P 1/14

(52) U.S. Cl. CPC A61K 36/82; A61P 1/14; A61K 2236/15; A61K 2236/19; A61K 2236/51; A61K 2236/53

(57) ABSTRACT

The present invention discloses an etiolated green tea polysaccharide, its extraction method and application. The method comprises: S1. crushing, defatting, decolorizing, and drying the tea; S2. ultrasonically mixing the powder with an extraction liquid, adding a complex enzyme to obtain a first mixture; S3. filtering, collecting the supernatant, and concentrating via rotary evaporation to obtain a second mixture; S4. precipitating with ethanol, centrifuging, and redissolving the precipitate to obtain a third mixture; S5. adding Sevage reagent for deproteinization, then concentrating; S6. dialyzing and drying to obtain the polysaccharide. In the present invention, the enzymatic extraction step and ultrasonic extraction step are performed simultaneously. By adjusting relevant processing parameters, the two extraction methods can effectively cooperate with each other, significantly improving the extraction efficiency of the tea polysaccharide.

### CROSS-REFERENCE TO RELATED APPLICATIONS

This application claims priority to Chinese Patent Application No. 202411806105.6, filed on Dec. 10, 2024, which is hereby incorporated by reference in its entirety.

### TECHNICAL FIELD

The present invention relates to the technical field of active ingredient extraction, specifically to an etiolated green tea polysaccharide and its extraction method and application.

### BACKGROUND

Tea polysaccharide (TPS) is a type of macromolecular active substance composed of various monosaccharides, uronic acids, proteins, etc., from tea leaves, which has multiple beneficial effects on human health, such as anti-obesity activity, blood glucose-lowering activity, lipid-lowering activity, etc.

Current main methods for extracting tea polysaccharide include hot water extraction, enzymatic hydrolysis extraction, and ultrasonic extraction. Among these, hot water extraction is low-cost and simple to operate but time-consuming and labor-intensive. Enzyme-assisted extraction can effectively catalyze the degradation of cell walls, allowing plant cells to release intracellular polysaccharide. Thus, this method enables the extraction of tea polysaccharide at a relatively low cost. In the prior art, ultrasonic extraction is often combined with heat extraction to shorten the extraction time of tea polysaccharide and improve the extraction yield of tea polysaccharide, etc.

Zhonghuang No. 1, a novel cultivar of Camellia sinensis (L.) O. Ktze, etiolated tea leaves are a high-quality variety suitable for producing high-end green tea, containing various nutrients beneficial to human health, such as theanine, tea polysaccharides, tea polyphenols, catechin polymers, etc. However, the yield per mu of Zhonghuang No. I etiolated tea leaves is limited, and their price is often high, which has prevented their widespread promotion and application. For such high-priced tea leaves, how to improve the extraction rate of nutrients therein and thereby enhance the utilization efficiency of the tea leaves has become a problem that needs to be solved.

### SUMMARY

The technical problem to be solved by the present invention is to provide a method for extracting etiolated green tea polysaccharide. This extraction method is adapted for the extraction of etiolated green tea, significantly improving the extraction rate of tea polysaccharide from the tea by combining enzymatic hydrolysis extraction with ultrasonic extraction, thereby enhancing the utilization rate of the tea leaves as raw material, which is conducive to the promotion and use of the tea.

The present invention is achieved through the following technical solution:
A method for extracting polysaccharide from etiolated green tea, comprising: S1. crushing etiolated green tea, followed by defatting and decolorizing, and drying for later use; S2. in an ultrasonic device, mixing etiolated green tea powder with an extraction liquid to obtain an extraction mixture, then adding a complex enzyme containing cellulase and papain to the extraction mixture for extraction, to obtain a first mixture; S3. filtering the first mixture, taking the supernatant, and concentrating by rotary evaporation to obtain a second mixture; S4. adding the second mixture to ethanol for precipitation, followed by centrifuging, taking the precipitate, and adding water for re-dissolution to obtain a third mixture; S5. adding Sevage reagent to the third mixture to remove proteins, obtaining a deproteinized mixture, then performing rotary evaporation on the deproteinized mixture to remove the Sevage reagent and concentrating to obtain a concentrate; S6. dialyzing and drying the concentrate to obtain etiolated green tea polysaccharide.

As a further improvement of the present invention, in S2, the etiolated green tea powder and the extraction liquid are mixed in a liquid-to-material ratio of 1:(10˜20) to obtain the extraction mixture.

As a further improvement of the present invention, in S2, 3˜7% of the complex enzyme is added to the extraction mixture, wherein the mass ratio of cellulase to papain in the complex enzyme is (2.5˜3.5): 1.

As a further improvement of the present invention, in S2, the extraction is performed under conditions of an extraction temperature of 60˜80° C. and an extraction pH of 4.0˜5.5 for 2˜2.5 hours to obtain the first mixture.

As a further improvement of the present invention, in S2, the ultrasonic frequency of the ultrasonic device is 80 kHz.

As a further improvement of the present invention, in S4, the volume ratio of the deproteinized mixture to the ethanol is 1:(4˜5).

As a further improvement of the present invention, in S5, the volume ratio of chloroform to n-butanol in the Sevage reagent is (4˜5): 1.

As a further improvement of the present invention, in S1, after crushing the etiolated green tea, it is passed through an 80˜100 mesh sieve to obtain the etiolated green tea powder, and the etiolated green tea powder is soaked in 95% ethanol for defatting and decolorizing, followed by natural air drying.

In a second aspect, the present invention provides an etiolated green tea polysaccharide prepared by any one of the aforementioned extraction methods for etiolated green tea polysaccharide.

In a third aspect, the present invention provides an application of the aforementioned etiolated green tea polysaccharide in the field of pharmaceuticals for regulating intestinal flora and assisting in weight loss.

In the prior art, ultrasonic extraction and enzymatic hydrolysis extraction methods are widely used in the extraction processes of natural plant polysaccharides. While there are cases where the two methods are used sequentially, instances of their simultaneous use are relatively rare. The extraction method described in the present invention can rapidly and efficiently disrupt the cell walls of etiolated green tea, allowing the active components therein to be released as much as possible.

In the present invention, on one hand, complex enzymes are used to decompose cellulose and hemicellulose in the cell walls, enabling the tea polysaccharide encapsulated within the cell walls to be released as much as possible; on the other hand, the cavitation, thermal, and mechanical effects of ultrasonic are utilized to assist enzymatic hydrolysis. Specifically, the present invention subjects the etiolated green tea powder to ultrasonic treatment simultaneously with enzymatic hydrolysis using cellulase and papain. When ultrasonic waves act on tea cells, the tea cells are disrupted and torn, forming numerous small cavities. These small cavities are torn open and then instantaneously close, generating instantaneous pressures as high as several thousand atmospheres during the process. This causes the cell walls and organisms in the tea powder to rupture instantly, forcing the tea polysaccharide to be released and flow out. Meanwhile, cellulase and papain, which decompose cellulose and proteins in the tea cells, can also enter the cavities, further accelerating the decomposition of cellular tissues. This facilitates the release of tea polysaccharide, thereby significantly shortening the extraction time and improving the extraction efficiency of tea polysaccharide.

The etiolated green tea polysaccharide prepared by the aforementioned extraction method can exist in a low molecular weight form. Low molecular weight polysaccharide can better retain their original activity and are more easily absorbed by cells. Meanwhile, the inventors discovered through experiments that the extracted etiolated green tea polysaccharide increases the relative abundance of beneficial bacterial genera in the intestinal flora, such as Bacteroides, Lactococcus, and Faecalibacterium, while also reducing the relative abundance of conditional pathogenic genera such as Escherichia Shigella and Dorea. Therefore, the inventors have confirmed that etiolated green tea polysaccharide can promote the homeostasis of gut microbiota and inhibit obesity by regulating human intestinal flora, thereby assisting in weight loss.

### BRIEF DESCRIPTION OF THE DRAWINGS

The following figures are provided to be used in conjunction with the preferred embodiments of the present invention to aid in understanding the objectives and advantages of the invention, wherein:

FIG. 1 is a statistical chart combining the test results of etiolated green tea polysaccharide extraction rate in Comparative Example 1 with those in Embodiment 1.

FIG. 2 is a statistical chart combining the test results of etiolated green tea polysaccharide extraction rate in Comparative Example 2 with those in Embodiment 1.

FIG. 3 is a statistical chart combining the test results of etiolated green tea polysaccharide extraction rate in Comparative Example 3 with those in Embodiment 1.

FIG. 4 is a statistical chart combining the test results of etiolated green tea polysaccharide extraction rate in Comparative Example 4 with those in Embodiment 1.

FIG. 5 is a statistical chart combining the test results of etiolated green tea polysaccharide extraction rate in Comparative Example 5 with those in Embodiment 1.

FIG. 6 is a composite diagram of molecular weight and IR spectrum of etiolated green tea polysaccharide (GTP).

FIG. 7 is a composite diagram of Congo red test, scanning electron micrograph, and XRD pattern of GTP.

FIG. 8 is a statistical chart of pH and OD600 values during in vitro fermentation.

FIG. 9 is a statistical chart of the effects of etiolated green tea polysaccharide on intestinal flora ASV.

FIG. 10 is a sequencing curve of intestinal flora.

FIG. 11 is a NMDS analysis chart of faecal fermentation broth microbiota among different groups.

FIG. 12 is a statistical chart of the effects of GTP on intestinal flora at the phylum level.

FIG. 13 is a statistical chart of the effects of GTP on intestinal flora at the genus level.

FIG. 14 is a statistical chart of LEfSe analysis results among intestinal flora groups.

### DESCRIPTION OF EMBODIMENTS

The present invention is further described in detail below according to the figures and embodiments.

To make the objectives, technical solutions, and advantages of the present invention clearer, the following describes the invention in further detail with reference to the figures and embodiments. It should be understood that the specific embodiments described herein are only intended to explain the invention and not all possible embodiments. Based on the embodiments of the present invention, all other embodiments obtained by a person of ordinary skill in the art without creative efforts shall fall within the protection scope of the present invention.

### Embodiment 1

This embodiment provides a method for extracting etiolated green tea polysaccharide, comprising the following steps:
S1. Crush etiolated green tea and sieve it through an 80-mesh sieve to obtain etiolated green tea powder. Soak the etiolated green tea powder in 95% ethanol for 2 hours for decolorizing and defatting, then remove and air-dry naturally for later use;
S2. In an ultrasonic device, under conditions of 80 kHz ultrasonic frequency, 80° C. extraction temperature, and pH 5.5, prepare an extraction mixture by combining etiolated green tea powder with an extraction liquid at a liquid-to-material ratio of 1:20 g/mL. Then, add 7% complex enzyme to the extraction mixture, mix for 2.5 hours to obtain the first mixture. The complex enzyme includes cellulase and papain, with a mass ratio of 3:1;
S3. Filter the first mixture, take the supernatant, and concentrate it by rotary evaporation to obtain the second mixture;
S4. Pour the second mixture into ethanol for precipitation, with a volume ratio of deproteinized mixture to ethanol of 1:4. After precipitation, centrifuge the mixture, take the precipitate, and redissolve it in water to obtain the third mixture;
S5. Add Sevage reagent to the third mixture to remove proteins, obtaining a deproteinized mixture. Then, perform rotary evaporation on the deproteinized mixture to remove the Sevage reagent and concentrate it to obtain a concentrate. The Sevage reagent includes chloroform and n-butanol, with a volume ratio of 4:1;
S6. Dialyze the concentrate with deionized water, then dry it to obtain etiolated green tea polysaccharide (hereinafter referred to as GTP).

Preferably, in S5, a vacuum freeze-drying equipment is used for drying to obtain the etiolated green tea polysaccharide. This method can reduce the destruction of active substances in the etiolated green tea polysaccharide during the drying step.

### Comparative Example 1

This comparative example differs from Embodiment 1 in that, in this example, the extraction step in S2 is performed at extraction temperatures of 40° C., 50° C., 60° C., and 70° C., respectively.

### Comparative Example 2

This comparative example differs from Embodiment 1 in that, in this example, the extraction mixture is prepared at liquid-to-material ratios of etiolated green tea powder to extraction liquid of 1:10, 1:15, 1:25, and 1:30 g/mL, respectively, for the extraction step in S2.

### Comparative Example 3

This comparative example differs from Embodiment 1 in that, in this example, the extraction step in S2 is performed at extraction pH values of 4, 4.5, 5, and 6, respectively.

### Comparative Example 4

This comparative example differs from Embodiment 1 in that, in this example, the extraction step in S2 is performed at complex enzyme addition concentrations of 1%, 3%, 5%, and 9%, respectively.

### Comparative Example 5

This comparative example differs from Embodiment 1 in that, in this example, the extraction duration in S2 is 1 h, 1.5 h, 2 h, and 3 h.

### Comparative Example 6

Conventional ultrasonic extraction method was used to extract etiolated green tea polysaccharide from the same mass of etiolated green tea powder as in Embodiment 1.

### Comparative Example 7

Conventional enzymatic extraction method was used to extract etiolated green tea polysaccharide from the same mass of etiolated green tea powder as in Embodiment 1.

### Results Testing:

### I. Detection of Etiolated Green Tea Polysaccharide Extraction Yield:

The extraction rates of GTP extracted in Embodiment 1 and Comparative Examples 1-5 were detected respectively. This detection step first requires weighing the etiolated green tea powder before extraction to obtain the “mass of the original tea sample”. Then, the phenol-sulfuric acid method was primarily used to detect the content of the extracted GTP, obtaining the “mass of tea polysaccharide in the extract”. Finally, the etiolated green tea polysaccharide extraction rates were calculated using the formula below.

$$Extraction Rate ( \% ) = ( \frac{Mass of Tea Polysaccharide in the Extract}{Mass of the Original Tea Sample} ) \times 100 \%$$

### II. Detection of Structural Characterization of Etiolated Green Tea Polysaccharide:

The total sugar content in GTP prepared in Embodiment 1 was determined using the phenol-sulfuric acid method; the reducing sugar content in GTP was determined using the dinitrosalicylic acid method; the protein content in GTP was determined using the BCA method; the uronic acid content in GTP was determined using the m-hydroxydiphenyl method; the molecular weight of GTP was determined using high-performance liquid chromatography.

Simultaneously, infrared spectrometer was used to analyze the infrared spectrum of the polysaccharide, scanning electron microscope was used to analyze the microstructure of the polysaccharide, Congo red assay was used to determine the triple-helix structure of the polysaccharide, and X-ray diffraction experiments were used to determine the presence of crystalline structures in the polysaccharide.

### III. Detection of the Impact of Etiolated Green Tea Polysaccharide on Intestinal Flora:

### A. Medium Preparation:

Precisely weigh 0.45 g KH₂PO₄, 0.45 g K₂HPO₄, 0.05 g NaCl, 0.064 g CaCl₂·2H₂O, 0.09 g MgSO₄·7H₂O, 2.5 g yeast extract, 10 g tryptone, 2 mL hemin (5 mg/mL), 1 g L-cysteine, 200 μL Vitamin I (prepared by dissolving 2 mg cyanocobalamin (VB₁₂), 2 mg biotin (VH), 10 mg folic acid, 6 mg p-aminobenzoic acid, 30 mg pyridoxamine (VB₆) in deionized water, made up to 40 mL), dissolve in deionized water, and make up to 1 L to prepare the medium.

### B. Test Group Preparation:

Divide the prepared medium into 6 portions, and under a nitrogen atmosphere, use a peristaltic pump to fill 5 mL into each 25 mL penicillin vial. After autoclaving, individual fermentation media are obtained. The experiment was designed with a total of 6 groups. The grouping and naming are shown in Table 1. Among them, the enzymatic hydrolysis polysaccharide group was supplemented with a corresponding amount of etiolated green tea polysaccharide; the inulin group served as a positive control group with added carbon source.

TABLE 1
In Vitro Fermentation Experimental Design Table

| Group | Grouping and Dosage | Name |
|---|---|---|
| Normal-weight | Blank Control Group (No additional | NC |
| Group | carbon source added) | |
| | Inulin Group (0.04 g inulin) | INL |
| | Enzymatic Hydrolysis Polysaccharide | NP |
| | Group (0.04 g enzymatic hydrolysis | |
| | crude polysaccharide) | |
| Obese Group | Blank Control Group (No additional | OC |
| | carbon source added) | |
| | Inulin Group (0.04 g inulin) | OIN |
| | Enzymatic Hydrolysis Polysaccharide | OP |
| | Group (0.04 g enzymatic hydrolysis | |
| | crude polysaccharide) | |

### C. Preparation of Human Faecal Fermentation Broth:

In this detection, 12 volunteers were recruited. All volunteers had no digestive system diseases, fasting blood glucose below 100 mg/dL (excluding pre-diabetes or diabetes), no smoking history, no current medication history, and had not taken antibiotics within the last 3 months. Volunteers were categorized based on body mass index (BMI): BMI<25 as the normal weight group (6 cases), BMI>25 as the overweight/obese group (OB) (6 cases). Faecal specimens were collected from the volunteers. The faecal specimens were diluted with sterilized phosphate-buffered saline (PBS) to obtain a 10% faecal suspension (w/v). After centrifuging the faecal suspension (speed: 300 rpm, temperature: 6° C., duration: 5 min), 0.5 mL of the supernatant was inoculated into the medium. After shaking well, exhaust gas was removed using a gas bag. The samples were placed in a 37° C. constant temperature anaerobic incubator and cultured for 24 h. At 0, 12, and 24 h time points after the start of fermentation, 1 mL of fermentation broth was taken to measure relevant indicators.

### D. pH and OD600 Value Determination of Fermentation Broth:

An appropriate amount of the fermented broth from each group was taken at different times and its pH value was measured using a pH meter.

For OD600 value detection of in vitro faecal fermentation, the spectrophotometer wavelength was set to 600 nm, then the absorbance of the sample was measured to obtain the OD600 value. This value is used to assess microbial growth, metabolic activity, and the progress of the fermentation process in the fermentate. By monitoring the OD600 values of in vitro faecal fermentate at different time points or under different treatment conditions, the growth of microbial communities under varying conditions can be understood, thereby evaluating the stability and efficiency of the fermentation.

### E. Determination of Short-Chain Fatty Acids (SCFAs):

(1) The samples obtained from each group were centrifuged at different times (speed: 12,000 rpm, duration: 4 min), 500 μL of supernatant was taken, and 100 μL of crotonic acid-metaphosphoric acid solution was added. After mixing thoroughly, the mixture was placed in a −80° C. freezer for acidification for 24 h. After acidification, the mixture was thawed at 4° C., centrifuge again (speed: 10,000 rpm), the supernatant was taken and passed through a 0.22 μm aqueous microporous filter membrane;
(2) Gas chromatography detection was performed on the fermentation broth. A DB-FFAP gas chromatography column (0.32 mm×30 m×0.5 μm) was used with crotonic acid as the internal standard substance for detection.

The operating conditions for the gas chromatograph were as follows: inlet temperature 250° C., column oven initial temperature 75° C., increased at 20° C./min to 180° C. and held for 1 min, then increased at 40° C./min to 220° C. and held for 1 min, FID detector temperature 250° C., injection volume 1 μL.

F. Total DNA Extraction and 16S rDNA High-Throughput Sequencing:

After 24 hours of fermentation, 1.5 mL of the fermentation broth was centrifuged, and the precipitate was used to extract genomic DNA. Following total DNA extraction from the samples, primers were designed as 338F (5′-ACTCCTACGGGAGGCAGCA-3′) and 806R (5′-GGACTACHVGGGTWTCTAAT-3′). PCR amplification was performed, followed by purification, quantification, and normalization to construct sequencing libraries. Qualified libraries were sequenced using the Illumina Novaseq 6000 platform. Sequencing data were analyzed using the online platform BMKCloud (https://www.biocloud.net). Beta diversity analysis was conducted using QIIME software, employing Non-Metric Multi-Dimensional Scaling (NMDS) based on the Bray-Curtis algorithm. Community structure diagrams were generated based on species at the phylum and genus levels. Linear discriminant analysis of the effect size (LEfSe) was used to compare gut microbiota with significant differences between groups, applying the Kruskal-Wallis test (p<0.05) and an LDA score >3.5 for screening. Histograms of LDA value distributions and evolutionary cladograms were plotted.

### Detection Results:

### I. Detection Results of Etiolated Green Tea Polysaccharide Extraction Rate:

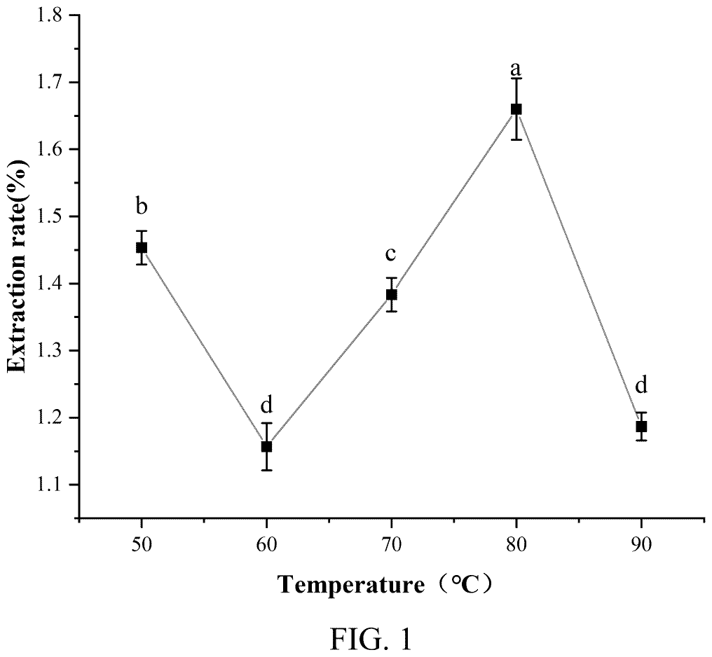

The statistical charts formed by combining the respective detection results of Comparative Examples 1˜5 with those of Embodiment 1 are shown in FIGS. 1˜5.

As shown in FIG. 1, when the extraction temperature was low, the inventors hypothesized that enzymatic extraction was predominant. Thus, when the temperature exceeded 50° C., enzyme activity likely decreased, leading to a reduction in polysaccharide extraction rate. Within the range of 60˜80° C., the extraction rate of etiolated green tea polysaccharide increased with rising temperature. The inventors speculated that ultrasonic extraction became dominant in this range, and the higher temperature facilitated substantial leaching of GTP, thereby enhancing the extraction rate. However, tests revealed that beyond 80° C., the extraction rate decreased as temperature increased, possibly because enzyme activity significantly declined above 80° C., preventing effective synergy with ultrasonic extraction. Consequently, only ultrasonic extraction was effective at this temperature, resulting in a reduced extraction rate.

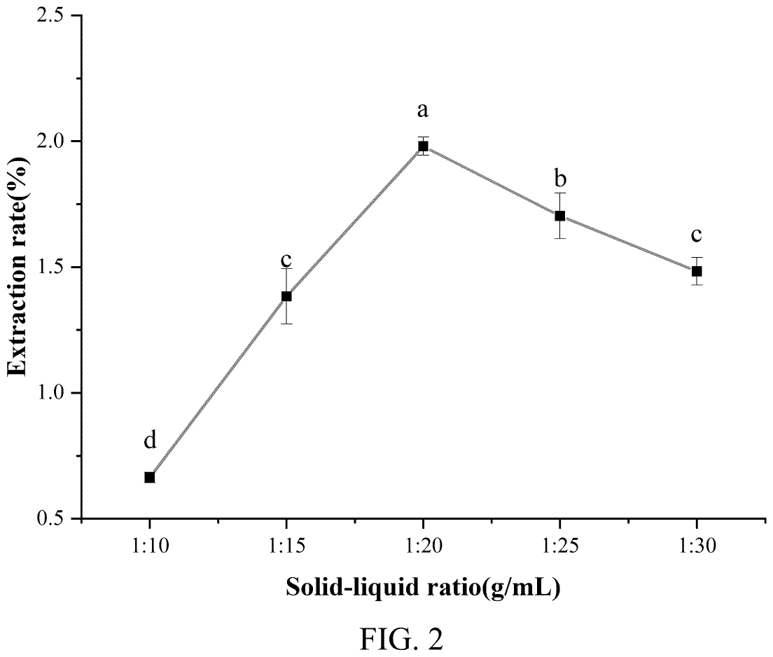

As illustrated in FIG. 2, within the liquid-to-material ratio range of 1:(10˜20) g/mL for etiolated green tea powder to extraction liquid, the polysaccharide extraction rate steadily increased, peaking at a ratio of 1:20 g/mL. The inventors attributed this to the increased extraction solvent volume, which expanded the contact area between the tea and solvent, promoting the dissolution of water-soluble polysaccharide. However, when the ratio reached 1:25 g/mL, the extraction rate declined with further increases in solvent. The inventors hypothesized that excessive solvent might affect the degree of polysaccharide gelation and its rheological properties, impairing mass transfer of the raw material.

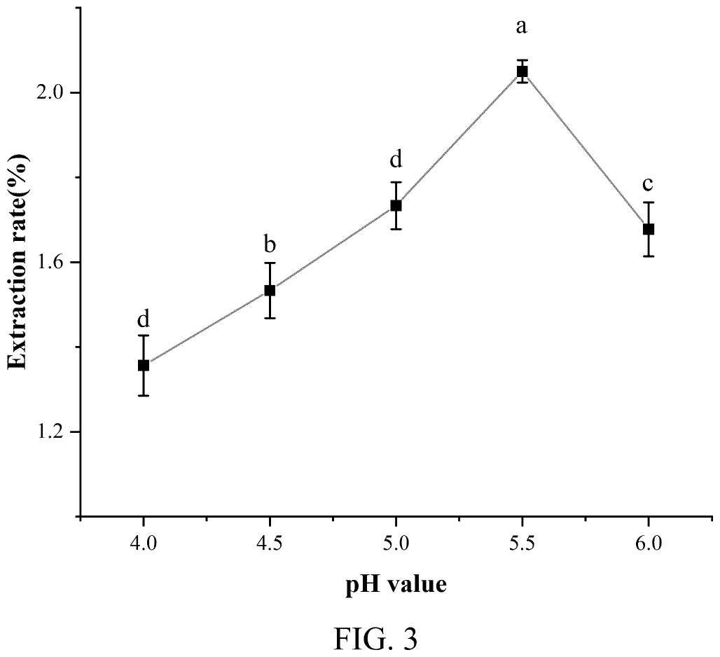

As shown in FIG. 3, as the extraction pH increased from 4.0 to 5.5, the polysaccharide extraction rate rose, reaching its maximum at pH 5.5. Beyond pH 5.5, the extraction rate decreased with further pH increase. The inventors suggested that higher pH may reduce enzyme activity, eliminating the synergistic assistance of enzymatic extraction and thus lowering the extraction rate.

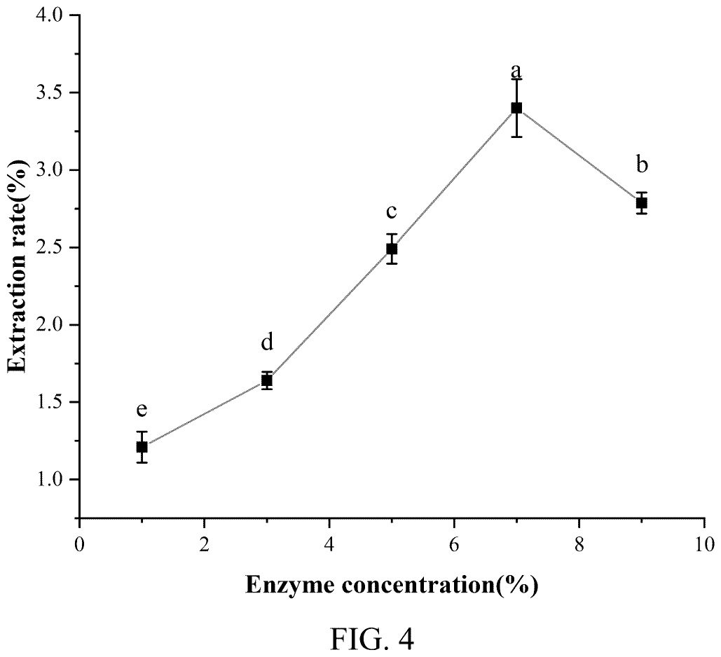

As depicted in FIG. 4, when the concentration of complex enzyme in the extraction mixture increased from 1% to 7%, the polysaccharide extraction rate improved, peaking at 7% enzyme concentration. However, beyond 7% enzyme concentration, the extraction rate decreased with further increases. The inventors speculated that this might be due to the enzymatic reaction reaching a threshold for substrate and enzyme concentration, or competitive inhibition by certain product-like substances affecting the reaction.

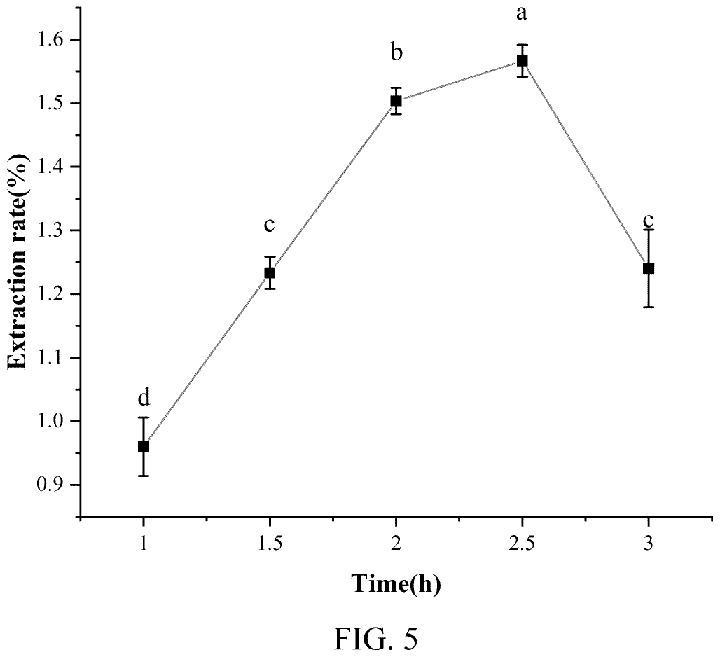

As shown in FIG. 5, the polysaccharide extraction rate increased with prolonged extraction time, reaching its maximum at 2.5 hours of enzymatic hydrolysis. However, when the extraction time was extended to 3 hours, the extraction rate decreased. The inventors hypothesized that excessive extraction time might lead to partial degradation of the etiolated green tea polysaccharide, reducing the total yield.

In Embodiment 1, the calculated extraction rate of etiolated green tea polysaccharide was 27.79%; in Comparative Example 6, it was 17.98%; and in Comparative Example 7, it was 21.54%. Thus, the method employed in this invention, through precise parameter control, facilitates the synergy between enzymatic and ultrasonic extraction, significantly enhancing the extraction efficiency of etiolated green tea and improving the utilization rate of the raw material.

### II. Detection Results of Etiolated Green Tea Polysaccharide Structural Characterization:

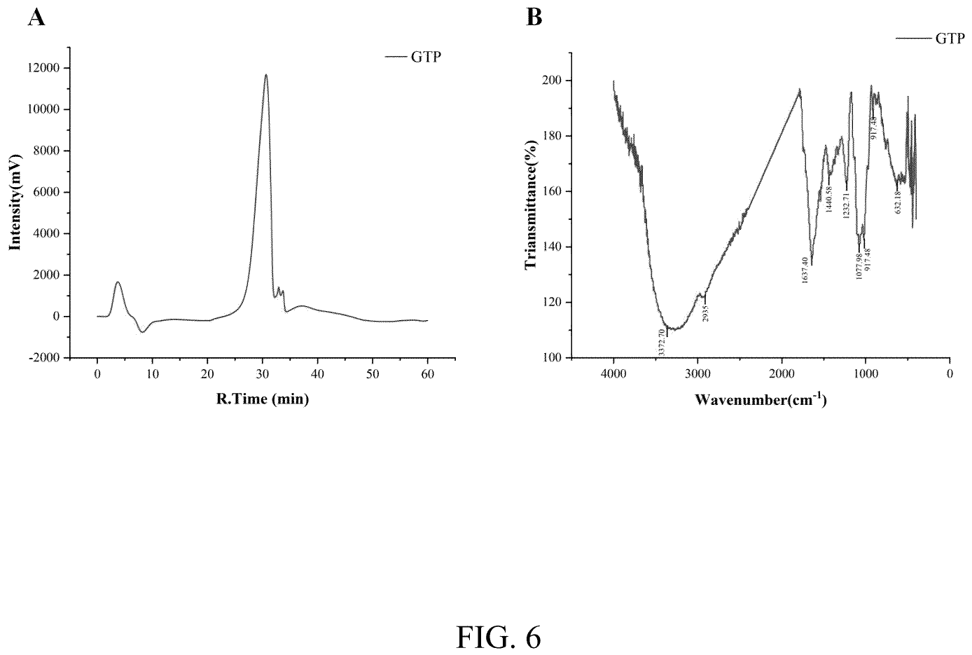

As shown in FIG. 6A, GTP has a relatively small molecular weight. The inventors speculated that enzymatic hydrolysis and ultrasonication may cause polysaccharide to exist in small molecular forms, which can retain biological activity and enhance cellular absorption rates. FIG. 6B shows the infrared spectrum of GTP. Analysis reveals characteristic peaks for O—H, C—H, —COOH, and C—O—C functional groups, indicating the presence of uronic acids and β-pyranose configurations in GTP.

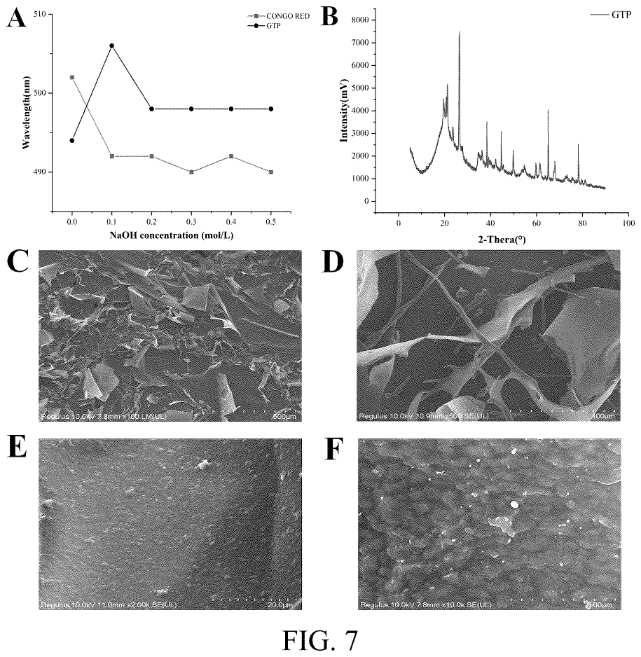

As shown in FIG. 7A, a triple-helix structure is present in GTP. The inventors hypothesized that this might be a prerequisite for the physiological activity of certain components in GTP. Crystal structure is commonly used to assess the crystalline properties of polysaccharide and predict its physical characteristics, such as flexibility, swelling capacity, and solubility. FIG. 7B displays the XRD diffraction pattern of GTP. The sharp and strong diffraction peak at 20=21.32° indicates the presence of large microcrystals in GTP. A narrow, sharp characteristic peak appears at 26.54°, confirming the crystalline structure of GTP. Slightly smaller diffraction absorption peaks in the 40° ˜50° range are associated with uronic acids in acidic polysaccharide. FIGS. 7C˜F show scanning electron microscope images of GTP. GTP exists as irregular flakes with distinct elongated chain-like structures and relatively compact spherical connections. The microstructure of etiolated green tea polysaccharide extracted using the synergistic ultrasonic-enzymatic hydrolysis technique in this invention differs significantly from previously studied tea polysaccharides. It exhibits a complex entangled structure, suggesting large internal voids and easy exposure of active regions, thereby endowing the polysaccharide with high physiological activity. The inventors speculated that this might be related to the degradation of macromolecular polysaccharides and the breakage of polymer molecular chains caused by ultrasonication and enzymatic action.

### III. Effects of Etiolated Green Tea Polysaccharide on Gut Microbiota:

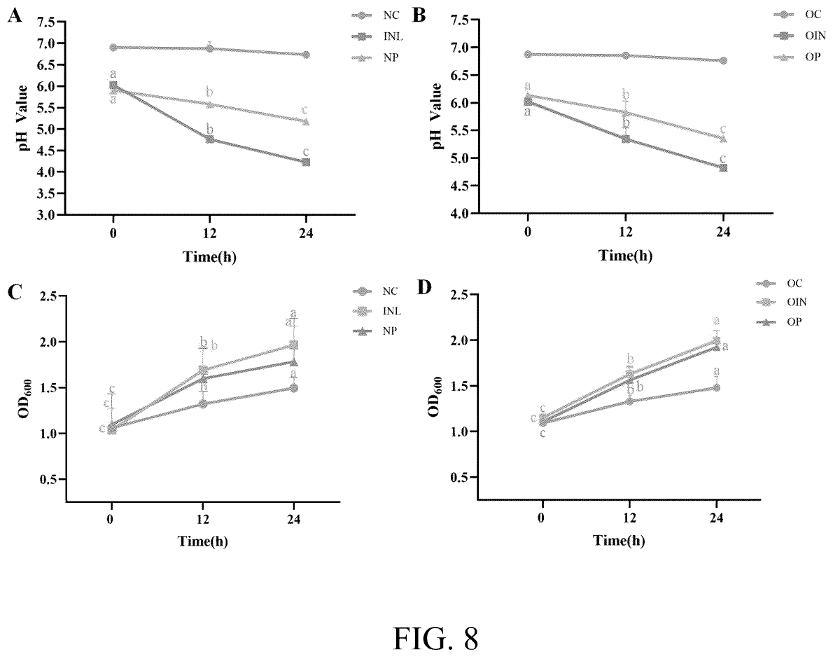

As shown in FIGS. 8A and B, there is almost no difference in pH between the NC and OC groups. The pH values of the NC and OC groups gradually decreased during fermentation. After adding GTP and INL, the pH decreased significantly (while p600 showed no significant difference (p>0.05)). After 12 hours of fermentation, OD600 increased significantly in all groups. The p600 of the NC and OC groups was higher than that of the NP and OP groups and continued to increase steadily. The OD600 of the NC and OC groups increased slowly throughout fermentation and was significantly lower than that of the NP and OP groups. The increase in p600values indicates that GTP addition increased microbial density in the fermentation broth and promoted the growth of gut microbiota.

TABLE 2
Statistical Table of SCFAs Content during Fermentation

0 h SCFAs (mmol/L)

| Group | | Acetate salt | Propionate | Isobutyrate | Butyrate | Isovalerate | Valerate | Total acid |
|---|---|---|---|---|---|---|---|---|
| Normal | NC | 0.52 ± 0.20 | 0.23 ± 0.02 [symbol] | 0.04 ± 0.02 [symbol] | 0.15 ± 0.0 [symbol] | 0.0 [symbol] ± 0. [symbol] | 0. [symbol] ± 0.00 [symbol] | 1.09 ± 0. [symbol] |
| group | INL | 0.68 ± 0.2 [symbol] | 0.27 ± 0.03 [symbol] | 0.02 ± 0.00 [symbol] | 0.14 ± 0.04 [symbol] | 0.0 [symbol] ± 0.00 [symbol] | 0.0 [symbol] ± 0.00 [symbol] | 1.17 ± 0. [symbol] |
| | NP | 1.00 ± 0.58 [symbol] | 0.4 [symbol] ± 0. [symbol] | 0.02 ± 0.01 [symbol] | 0.17 ± 0.07 [symbol] | 0.0 [symbol] ± 0.01 [symbol] | 0.02 ± 0.01 [symbol] | 1. [symbol] ± 0.11 [symbol] |
| Obese | OC | 0.68 ± 0.19 [symbol] | 0.27 ± 0.0 [symbol] | 0.02 ± 0.00 [symbol] | 0.14 ± 0.04 [symbol] | 0.05 ± 0.00 [symbol] | 0.0 [symbol] ± 0.00 [symbol] | 1.40 ± 0.41 [symbol] |
| group | OIN | 1.11 ± 0.28 [symbol] | 0.3 [symbol] ± 0.2 [symbol] | 0.02 ± 0.01 [symbol] | 0.1 [symbol] ± 0.0 [symbol] | 0.06 ± 0.0 [symbol] | 0.0 [symbol] ± 0.00 [symbol] | 1.74 ± 0.46 [symbol] |
| | OP | 1.15 ± 1. [symbol] | 0.43 ± 0.1 [symbol] | 0.02 ± 0.01 [symbol] | 0.2 [symbol] ± 0.0 [symbol] | 0. [symbol] ± 0.01 [symbol] | 0. [symbol] ± 0.01 [symbol] | 1.87 ± 0.33 [symbol] |
| | | 12 h SCFAs (mmol/L) | | | | | | |
| Group | | Acetate | Propionate | Isobutyrate | Butyrate | Isovalerate | Valerate | Total acid |
| Normal | NC | 4.0 [symbol] ± 0. [symbol] | [symbol] ± [symbol] | 0.02 ± 0.01 [symbol] | 0.7 [symbol] ± 0.24 [symbol] | 0.04 ± 0.01 [symbol] | 0.0 [symbol] ± 0.01 [symbol] | 6.73 ± 0. [symbol] |
| group | INL | [symbol] .06 ± 0.8 [symbol] | 0. [symbol] ± 0.4 [symbol] | 0.01 ± 0.00 [symbol] | 0.1 [symbol] ± 0.05 [symbol] | 0.03 ± 0.01 [symbol] | 0.01 ± 0.00 [symbol] | [symbol] .92 ± 1.33 [symbol] |
| | NP | 9. [symbol] ± [symbol] | 3.72 ± 1. [symbol] | 0.06 ± 0.03 [symbol] | 1. [symbol] ± 0.3 [symbol] | 0.2 [symbol] ± 0.0 [symbol] | 0.25 ± 0.0 [symbol] | 15.82 ± 3. [symbol] |
| Obese | OC | 7. [symbol] ± 2. [symbol] | 1.90 ± 0. [symbol] | 0.05 ± 0.02 [symbol] | 1. [symbol] ± 0.3 [symbol] | 0.1 [symbol] ± 0.12 [symbol] | 0. [symbol] ± 0.01 [symbol] | 11.09 ± 3.65 [symbol] |
| group | OIN | 8. [symbol] ± 1.57 [symbol] | 1.30 ± 0.75 [symbol] | 0.02 ± 0.01 [symbol] | 0.4 [symbol] ± 0.0 [symbol] | 0.03 ± 0.01 [symbol] | 0. [symbol] ± 0.02 [symbol] | 10.41 ± 1.00 [symbol] |
| | OP | [symbol] ± 2.11 [symbol] | [symbol] ± 0.1 [symbol] | 0.20 ± 0.10 [symbol] | 1. [symbol] ± 0.34 [symbol] | 0. [symbol] ± 0.0 [symbol] | 0.23 ± 0.04 [symbol] | 9.34 ± 2.45 [symbol] |

| | | 24 h SCFAs (mmol/L) | | | | | | |
|---|---|---|---|---|---|---|---|---|
| Group | | Acetate | Propionate | Isobutyrate | Butyrate | Isovalerate | Valerate | Total acid |
| Normal | NC | [symbol] ± 2. [symbol] | 1. [symbol] ± 0. [symbol] | 0.0 [symbol] ± 0.0 [symbol] | 0.23 ± 0.1 [symbol] | 0.12 ± 0.0 [symbol] | 0.0 [symbol] ± 0.0 [symbol] | [symbol] .94 ± 3.92 [symbol] |
| group | INL | 11.4 [symbol] ± 2. [symbol] | 5. [symbol] ± 1. [symbol] | 0.06 ± 0.02 [symbol] | 1.92 ± 0.29 [symbol] | 0.16 ± 0.0 [symbol] | 0.06 ± 0.0 [symbol] | 21. [symbol] 6 ± 13.69 [symbol] |
| | NP | 17.27 ± 5. [symbol] | 6.55 ± 1. [symbol] | 0.52 ± 0.10 [symbol] | 5.40 ± 2. [symbol] | 1.26 ± 0.37 [symbol] | 1.13 ± 0.34 [symbol] | [symbol] 2.22 ± 10.92 [symbol] |
| Obese | OC | [symbol] .46 ± 1.5 [symbol] | 2.57 ± 0. [symbol] | 0.0 [symbol] ± 0.02 [symbol] | 1.79 ± 0.49 [symbol] | 0.14 ± 0.04 [symbol] | 0.2 [symbol] ± 0.0 [symbol] | 14.2 [symbol] ± [symbol] .24 [symbol] |
| group | OIN | 14.9 [symbol] ± 2. [symbol] | 2.48 ± 0. [symbol] | 0.10 ± 0.04 [symbol] | 2.26 ± 1.02 [symbol] | 0.21 ± 0.0 [symbol] | 0.3 [symbol] ± 0. [symbol] | 20.34 ± 4.5 [symbol] |
| | OP | 11.44 ± 2.7 [symbol] | [symbol] ± 0.7 [symbol] | 0.27 ± 0.0 [symbol] | 2.69 ± 1.0 [symbol] | 0. [symbol] ± 0.14 [symbol] | 0.60 ± 0.23 [symbol] | 1 [symbol] .93 ± [symbol] .31 [symbol] |

[symbol]

indicates data missing or illegible when filed

Table 2 shows the SCFAs content at different fermentation times, used to evaluate the in vitro fermentation performance of GTP in feces from different populations and the utilization level of polysaccharide by gut bacteria. From Table 2, acetate had the highest content among all SCFAs produced by gut microbial fermentation of GTP, followed by propionate and butyrate. After adding GTP or INL to the normal-weight and obese groups, compared to the control groups, the total acid, acetate, and propionate contents increased significantly after 12 and 24 hours of fermentation.

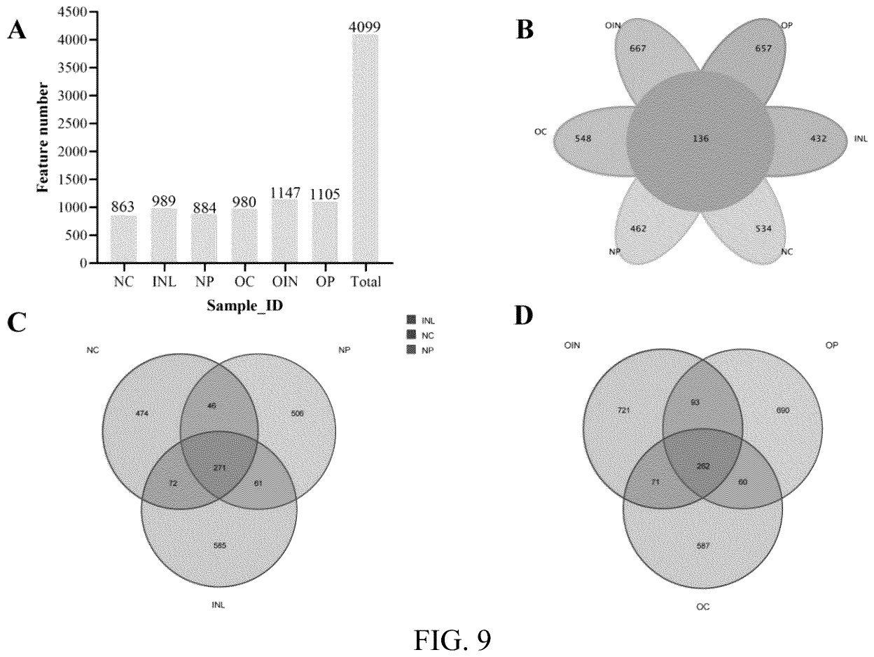

From FIG. 9A, a total of 4099 ASVs were obtained from the six groups, with 863 in the NC group and 980 in the OC group. As shown in FIG. 9B, the number of shared ASVs among the six groups was 136. By comparing the ASV numbers in the normal-weight and obese groups in FIGS. 9C and D, it can be seen that after adding inulin and GTP, the number of ASVs increased in all groups, especially in the obese group after GTP addition, where the number of ASVs increased significantly. Additionally, the number of characteristic ASVs was highest in the INL and OIN groups, followed by the NP and OP groups, indicating that polysaccharide addition significantly affected the gut microbiota.

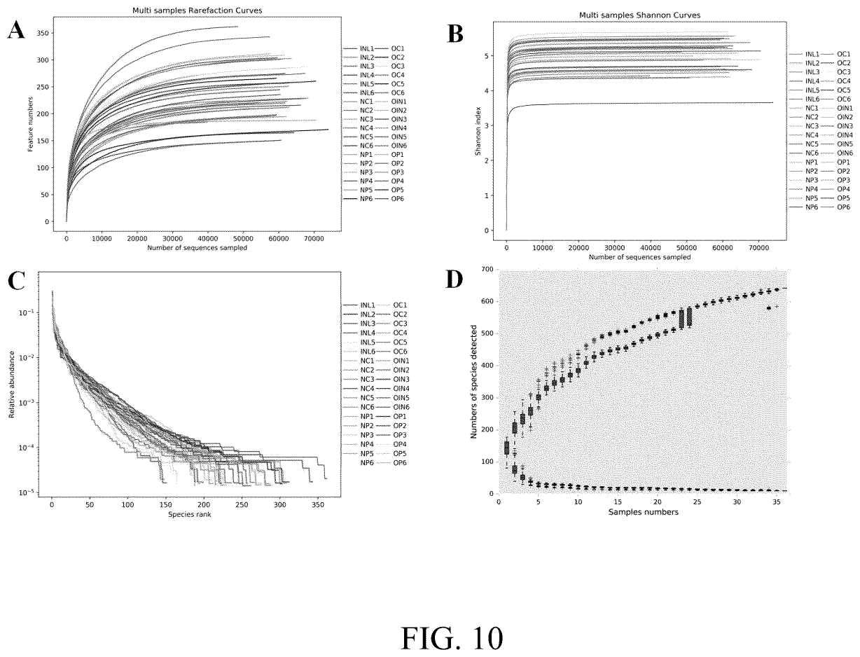

As shown in FIGS. 10A˜B, both the rarefaction curve and Shannon index curve plateau, reflecting the species diversity and richness in the samples and indicating that the vast majority of microbial species information has been covered. The sample sequences are sufficient, meeting the requirements for further analysis. As shown in FIG. 10C, there are significant differences in richness and evenness among the groups, indicating sufficient sequencing depth. As shown in FIG. 10D, the species accumulation curve plateaus during sequencing, indicating that the sequencing library is large enough to cover most bacterial diversity in all samples.

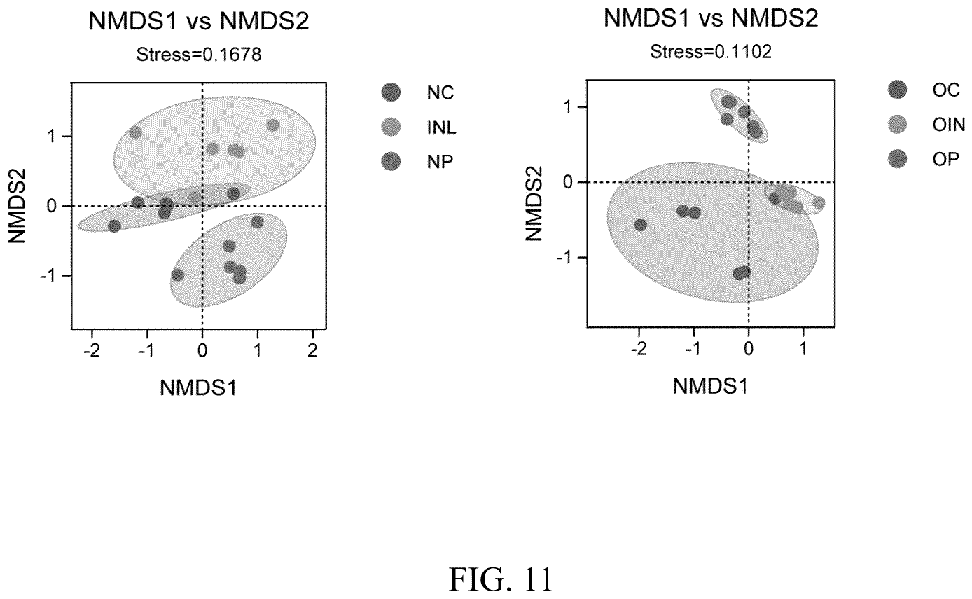

Principal component analysis was performed on the gut microbiota, as shown in FIG. 11. NMDS analysis revealed that the gut microbiota structures of GTP and INL differ in the fecal fermentation broth of normal-weight and obese individuals. This indicates that GTP addition alters the microbial composition in the human gut and shows certain differences compared to INL.

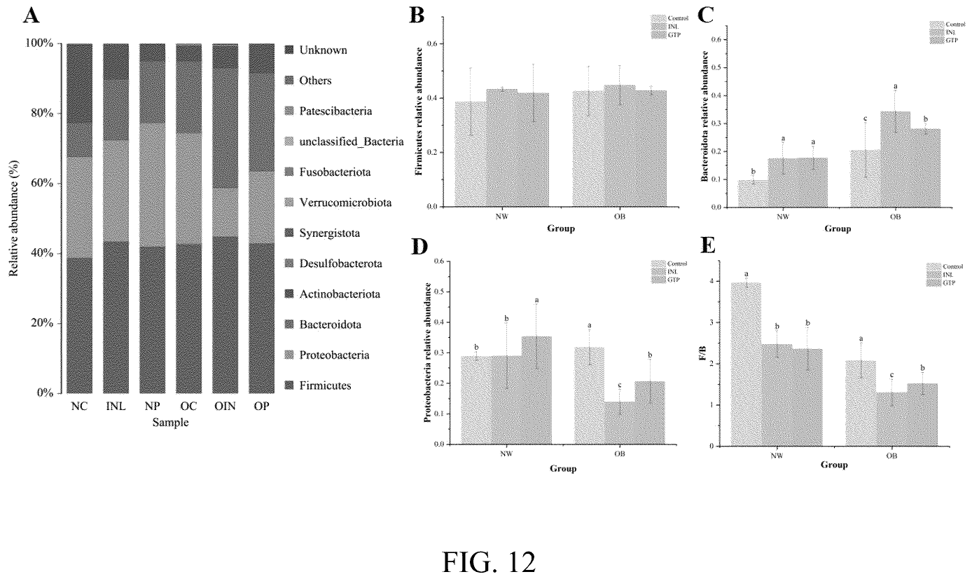

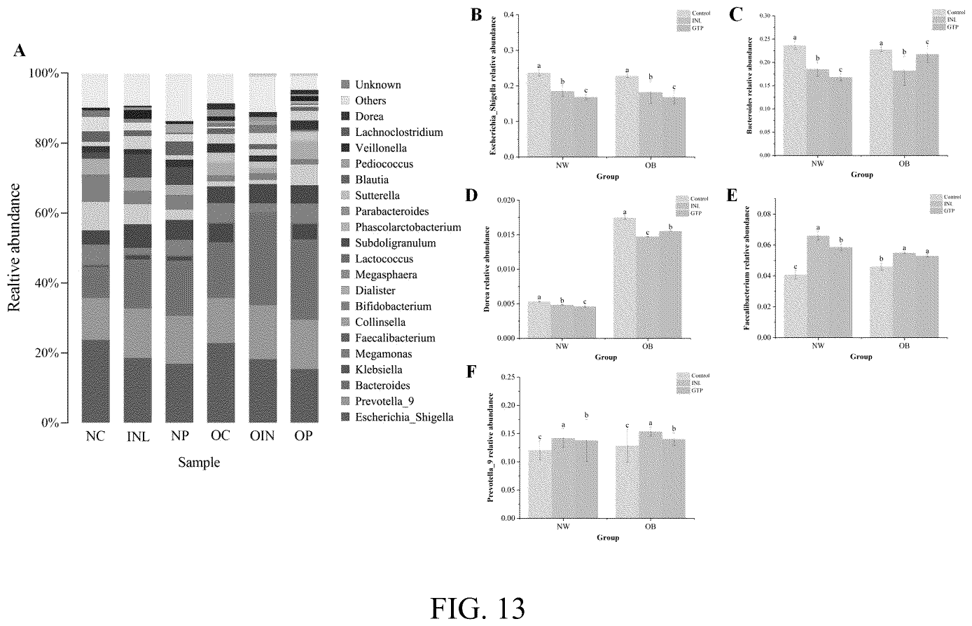

The top 10 most abundant phyla were evaluated at the phylum level (as shown in FIG. 12A), with the relatively abundant microbial phyla being Firmicutes, Bacteroidota, Proteobacteria, and Actinobacteriota. FIG. 12B shows that the addition of GTP and INL had little effect on Firmicutes, with no significant differences in relative abundance among the groups (p>0.05); from FIG. 12C, it can be observed that after adding GTP or INL, the relative abundance of Bacteroidota in the corresponding fermentation broth significantly increased (p<0.05). The Firmicutes-to-Bacteroidota ratio (F/B) can serve as an indicator of gut microbial imbalance and the degree of obesity. In both the NW and OB groups, the F/B ratio significantly decreased after the addition of GTP and INL (as shown in FIG. 12E), indicating that the fermentation substrates GTP and INL can inhibit obesity to some extent. As shown in FIG. 13, GTP increased the relative abundance of beneficial bacteria genera, such as Bacteroides, Lactococcus, and Faecalibacterium, while also reducing the relative abundance of harmful bacteria genera, such as Escherichia Shigella and Dorea.

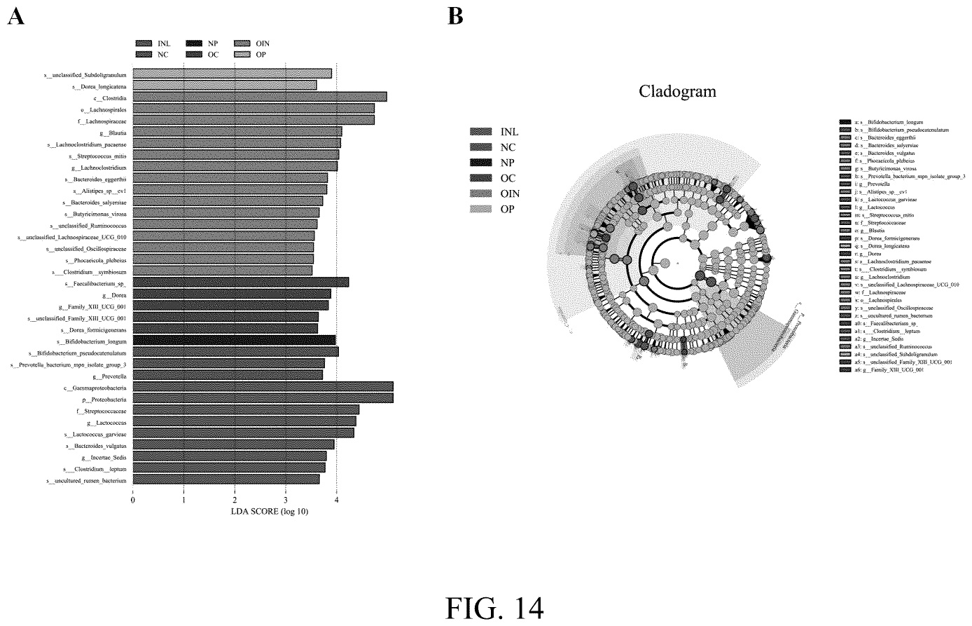

The distribution histogram and evolutionary cladogram of LDA values are shown in FIGS. 14A˜B. Species with LDA values (Log 10)>3.5 in each group were plotted as a bar chart. There were 3, 9, 1, 5, 16, and 2 species with significant differences in the NC, INL, NP, OC, OIN, and OP groups, respectively. The species evolutionary cladogram indicates significant differences in bacterial community distribution among the six groups. At the phylum level, Proteobacteria in the INL group was the species with the most significant differences compared to other groups. At the class level, Gammaproteobacteria in the INL group and Clostridia in the OIN group can serve as biomarkers with significant differences at this level, respectively. At the genus level, Blautiah and Lachnoclostridium in the OIN group, Dorea and FAMILY_XIII_UCG_001 in the OC group, Prevotella in the NC group, Lactococcus and Incertae Sedis in the INL group can serve as biomarkers with significant differences at this level. At the species level, unclassified_Subdoligranulum and Dorea longicatena in the OP group, and Bifidobacterium longum in the NP group can serve as biomarkers with significant differences at this level.

In summary, it can be concluded that etiolated green tea polysaccharide can benefit gut microbial homeostasis, inhibit obesity by modulating the gut microbiota in both normal and obese individuals, and is conducive to human health. Therefore, the inventors determined that etiolated green tea polysaccharide can be applied in the field of gut microbiota regulation and weight-loss-related medications.

Finally, it should be noted that the above embodiments are only used to illustrate the technical solutions of the present invention and are not intended to limit them. Although the present invention has been described in detail with reference to the aforementioned embodiments, those of ordinary skill in the art should understand that they can still modify the technical solutions described in the various embodiments or equivalently replace some of the technical features. These modifications or replacements do not cause the essence of the corresponding technical solutions to deviate from the scope of the technical solutions of the embodiments of the present invention.

What is claimed is:

1. Use of an etiolated green tea polysaccharide in the field of pharmaceuticals for regulating intestinal flora and assisting in weight loss is characterized in that the etiolated green tea polysaccharide is prepared through the following extraction method:
S1. crushing etiolated green tea, followed by defatting and decolorizing, and drying for later use;
S2. in an ultrasonic device, mixing etiolated green tea powder with an extraction liquid to obtain an extraction mixture, then adding a complex enzyme containing cellulase and papain to the extraction mixture for extraction, to obtain a first mixture;
S3. filtering the first mixture, taking the supernatant, and concentrating by rotary evaporation to obtain a second mixture;
S4. adding the second mixture to ethanol for precipitation, followed by centrifuging, taking the precipitate, and adding water for re-dissolution to obtain a third mixture;
S5. adding Sevage reagent to the third mixture to remove proteins, obtaining a deproteinized mixture, then performing rotary evaporation on the deproteinized mixture to remove the Sevage reagent and concentrating to obtain a concentrate;
S6. dialyzing and drying the concentrate to obtain etiolated green tea polysaccharide.

2. The Use of the etiolated green tea polysaccharide according to claim 1 in the field of pharmaceuticals for regulating intestinal flora and assisting in weight loss, characterized in that, in S2, the etiolated green tea powder and the extraction liquid are mixed in a liquid-to-material ratio of 1:(10˜20) to obtain the extraction mixture.

3. The Use of the etiolated green tea polysaccharide according to claim 2 in the field of pharmaceuticals for regulating intestinal flora and assisting in weight loss, characterized in that, in S2, 3˜7% of the complex enzyme is added to the extraction mixture, wherein the mass ratio of cellulase to papain in the complex enzyme is (2.5˜3.5): 1.

4. The Use of the etiolated green tea polysaccharide according to claim 3 in the field of pharmaceuticals for regulating intestinal flora and assisting in weight loss, characterized in that, in S2, the extraction is performed under conditions of an extraction temperature of 60˜80° C. and an extraction pH of 4.0˜5.5 for 2˜2.5 hours to obtain the first mixture.

5. The Use of the etiolated green tea polysaccharide according to claim 1 in the field of pharmaceuticals for regulating intestinal flora and assisting in weight loss, characterized in that, in S2, the ultrasonic frequency of the ultrasonic device is 80 KHz.

6. The Use of the etiolated green tea polysaccharide according to claim 1 in the field of pharmaceuticals for regulating intestinal flora and assisting in weight loss, characterized in that, in S4, the volume ratio of the deproteinized mixture to the ethanol is 1:(4˜5).

7. The Use of the etiolated green tea polysaccharide according to claim 1 in the field of pharmaceuticals for regulating intestinal flora and assisting in weight loss, characterized in that, in S5, the volume ratio of chloroform to n-butanol in the Sevage reagent is (4˜5): 1.

8. The Use of the etiolated green tea polysaccharide according to claim 1 in the field of pharmaceuticals for regulating intestinal flora and assisting in weight loss, characterized in that, in S1, after crushing the etiolated green tea, it is passed through an 80˜100 mesh sieve to obtain the etiolated green tea powder, and the etiolated green tea powder is soaked in 95% ethanol for defatting and decolorizing, followed by natural air drying.
